(19) United States
(12) Patent Application Publication
(10) Pub. No.: US 2017/0196726 A1
(43) Pub. Date: Jul. 13, 2017

(54) UNISEX EXTERNAL URINE COLLECTION APPARATUS

(71) Applicant: Jacqueline R. SanAntonio, Chesapeake, VA (US)

(72) Inventor: Jacqueline R. SanAntonio, Chesapeake, VA (US)

(21) Appl. No.: 14/990,831

(22) Filed: Jan. 8, 2016

(51) Int. Cl. A61F 5/44

(52) U.S. Cl. CPC A61F 5/4404; A61F 5/4408; A61F 5/4405

(57) ABSTRACT

An external urine collection apparatus for use by either male or female wearers has an elongated cone-like collection apparatus having a conformable seal around a top opening that appears as a convex curve when viewed from the side. Strap attachment loops are disposed circumferentially around the outside surface. Support straps each have a proximal end selectively attached to the attachment loops. Each strap has a distal end that is adjustably attached to a waist-encircling strap or belt to hold the urine collection apparatus in sealed contact with the wearer's genital region. A threaded connection at the lower terminus of the elongated cone-like collection apparatus accepts a detachable drainage tube which may be connected to well-known collection bags or the like to accumulate the urine. One or more reflux valves may be located adjacent the lower terminus or in the detachable drainage hose.

### FIELD OF THE INVENTION

The invention pertains to an external urine collection apparatus and, more particularly, to a urine collection apparatus adapted for use by either a male or female wearer.

### BACKGROUND OF THE INVENTION

Urine collection devices and/or incontinence wear for both male and female persons both well-known and widely used. Incontinence pads, briefs, diapers, etc. are used externally to absorb excreted urine. Use of these products may result in wet skin, odor, potential skin breakdown, and potential infection.

Internal urine collection devices are also available. Such devices include catheters including so-called “straight” catheters typically for intermittent use and indwelling “Foley” catheters typically for longer term use.

Straight catheters are typically inserted into the bladder via the urethra, the urine is drained from the bladder, and then the catheter is withdrawn. Typically a straight catheter may be used to empty a person's bladder when that person is unable to do so without aid. Typically patients may be taught to straight catheterize themselves.

Foley catheters have a retention balloon at the distal end that, after inflation, retains the distal end of the catheter within the bladder. Consequently, this may be used for longer term use and provides the benefit of continuous emptying of the bladder. A Foley catheter must be changed frequently to minimize urinary tract infections (UTIs) and may require professionally trained personnel to place and/or remove them.

Both types provide pathways for infection and have other possible consequences.

There are many external devices for use by both men and women ranging from single-use urinals to elaborate external, extra-labial devices for women, condom catheters for men, etc. However, all suffer from problems of effectivity in urine collection, problems with leakage, and especially problems with patient comfort.

It would, therefore, be advantageous to provide an external urine collection apparatus that may be comfortably worn by both men and women that may be worn for long periods of time comfortably with little risk of medical consequences arising from its use.

### DISCUSSION OF THE RELATED ART

Several attempts to provide urine collection device may be found in the prior art, for example U.S. Pat. No. 2,796,864 for SANITARY URINARY APPLIANCE issued Jun. 25, 1957 to Albert Johnson provides an oval urine collecting funnel supported on the patient by a waist-surrounding belt. A discharge portion of the funnel allows collected urine to flow out of the funnel into an attached collection receptacle.

U.S. Pat. No. 3,349,768 for PORTABLE URINAL PROVIDED WITH SUCTION MEANS FOR USE IN MICTURITION issued Oct. 31, 1967 to Francis Xavier Keane provides a portable apparatus for use in micturition, and more particularly, an apparatus adapted for collecting and conveying urine and other discharges from the region of the external genitalia. The device has a suction head shaped for releasable positioning in contact with female or male external genitalia, an inlet in the suction head for receiving any discharge from the genitalia, and a suction head outlet in communication with the inlet.

U.S. Pat. No. 4,013,077 for COLLECTING DEVICE issued Mar. 22, 1997 to Michael C. Ritota, et al. provides a collecting device for the collection of excretions from various body openings, both natural and surgical in nature, is disclosed. The collecting device includes an inflatable collar which may be inserted into a body opening prior to inflation, and then be inflated in order to firmly anchor itself within that opening and prevent its inadvertent removal therefrom, or attached to a body opening or appendage. A solid elongated tubular member is heat-sealed to either the inner diameter or outer diameter of the inflatable collar, and forms a continuous channel connecting the inflatable collar to a bag disposed external to the body opening so that body secretions from that opening are collected in the bag in a simple and hygienic manner.

U.S. Pat. No. 4,202,058 for FEMALE URINAL issued May 13, 1980 to Robert W. Anderson teaches a urinal for supine use. The urinal has a flexible inner liner cooperating with a rigid outer shell to define a generally oval-shaped receiving chamber. Edge portions of the inner liner protrude beyond the outer shell and are deformable to sealingly engage body parts of an intended user. A pump communicates through a duct section with the receiving chamber to evacuate collected liquid from the receiving chamber.

U.S. Pat. No. 4,692,160 for URINE COLLECTOR FOR INCONTINENT WOMEN HAVING ZIG•ZAG WALLS issued Sep. 8, 1987 to Max Nussbaumer discloses a two part urine collector comprised of a slightly elastic material having a collector structure arranged above a collecting cavity provided with an outlet. The collector structure is comprised of two side pieces in the form of zigzag-shaped prominences. The structure delimits a wet zone, while a moist zone is delimited by the lateral regions of the border together with a rear bead and a front bead. The collector structure is placed between the labia minora and labia majora of the woman, while the elements delimiting the moist zone come to lie outside the labia majora. The inventive urine collector enables incontinent women to freely move about without risk of their garments being contacted with urine.

U.S. Pat. No. 5,632,736 FLUID VOIDING APPARATUS issued May 27, 1997 to James C. Block provides an extra-labia urine voiding apparatus including a container sized and shaped to externally cover a vulval region of a female anatomy. The container has an open side and defines a reservoir that is adapted for receiving urine. The container also includes a flange having a substantially planar surface adapted to face and generally surround a periphery of the vulval region. An extra-labia sealing structure is affixed to the planar surface of the flange. The sealing structure includes a pressure sensitive adhesive adapted to provide a generally fluid tight primary adhesive seal between the container and external tissue generally surrounding the vulval region. The voiding apparatus also includes a conduit in fluid communication with the reservoir of the container and adapted for draining urine from the reservoir.

U.S. Pat. No. 5,735,835 for FEMALE EXTERNAL URINARY COLLECTION POUCH issued Apr. 7, 1998 to Marian J. Holland shows a female urinary collection device including a receptacle pouch for collecting urine. The receptacle pouch includes a first area for securing two strips of tape, one to each buttock of a user to create a relatively rigid engagement of the device with the body. In the upper zone, there is a stretch loop and foam belt for securing the pouch around the waist. Inside the receptacle pouch, there is a reservoir of adsorbent material with a wick which exits the receptacle pouch through a tubing connected to an orifice in the receptacle. The receptacle pouch fits around the labia majora of the user.

U.S. Pat. No. 6,299,606 URINE COLLECTION DEVICE issued Oct. 9, 2001 to Michael J. Young discloses a urine collection device for women has a bowl with sides that bow inwardly to provide a comfortable fit while minimizing spillage. The bowl has an aperture through which urine can pass to a removable specimen jar. An elongated handle extends from the front of the bowl, and is provided with finger grips on its bottom edge. The device can be molded from plastic, permitting it to be sanitized and reused.

U.S. Pat. No. 7,476,219 for URINARY INCONTINENCE DEVICE issued Jan. 13, 2009 to Oumeima Ben Youssef shows an external female incontinence device. The device comprises a flexible elastic strip having an opening in the strip, a fluid collection means surrounding the opening on one face, the strip and opening are sized and shaped so that when stretch fitted over the external urogenital organs the labia minor extends through the opening and a fluid tight fit between the upper face of the strip and the flesh surrounding the labia minora is formed. The device may further comprise a waistband for attachment of the device and to assist in maintaining a positive tension to press the device against the flesh surrounding the labia minora.

U.S. Pat. No. 8,235,956 for PACKAGING AND KIT FOR A FEMALE URINE VOIDING APPARATUS issued Aug. 7, 2012 to James C. Block discloses packaging for an extra-labial fluid voiding apparatus including a tube having a cylindrical wall, a closed end, and an open end. The cylindrical wall and closed end define a cavity that can contain the voiding apparatus. The cylindrical wall is pliable and has an inner diameter. A cap is adapted to respectively close and open the tube at the open end. The cap has an end plate with an annular flange extending therefrom. The annular flange is rigid and has an outer diameter. The annular flange is insertable into the cavity at the open end of the tube and within the inner diameter of the cylindrical wall. The outer diameter of the flange is larger than the inner diameter of the cylindrical wall to a degree such that the outer diameter and the inner diameter are engageable in a press-fit arrangement, when the cap is connected to the tube.

U.S. Pat. No. 8,337,477 for APPARATUS FOR EXTRA-LABIAL URINE VOIDING issued Dec. 25, 2012 to Mark Parks, et al. shows an extra-labia urine voiding apparatus including a container sized and shaped to externally cover a vulval region of a female anatomy. The container has an open side and defines a reservoir that is adapted for receiving urine. The container also includes a substantially planar surface disposed at a top of the container adapted to face and generally surround a periphery of the vulval region. An extra-labial sealing structure is affixed to the planar surface of the top of the container. The sealing structure includes a pressure sensitive adhesive adapted to provide a generally fluid tight primary adhesive seal between the container and external tissue generally surrounding the vulval region. A blocking mechanism connected proximate the top of the container prevents escape of urine from the voiding apparatus and contact of urine with the vulval region of the female anatomy. The voiding apparatus also includes a conduit in fluid communication with the reservoir of the container and adapted for draining urine from the reservoir.

U.S. Published Patent Application No. 2012/0103347 for URINARY INCONTINENCE DEVICE AND METHOD published May 3, 2012 upon application by Amy Wheaton, et al. discloses a urinary incontinence device comprising a flexible but shape-retaining body portion wherein the body portion has a longitudinal axis extending from an anterior end to a posterior end thereof. The body portion is elongated in the direction of the longitudinal axis and has a length and width sufficient to cover the vulva or retracted penis of a human female or male and to form a urine-receiving receptacle. The receptacle has shallow urine directing portion sloping inwardly and downwardly away from the vulva or retracted penis, and a deeper, narrower central urine collection portion having a urine outlet. The receptacle is elongated in the direction of the longitudinal axis of the body portion. The device also includes an attachment flange having an anterior end and a posterior end corresponding to the anterior end and the posterior end of the body portion, respectively. The attachment flange is formed to extend outwardly away from the body portion, and it includes an adhesive thereon for securing the device to entirely cover the vulva or retracted penis of a human female or male.

None of the patents and published patent applications, taken singly, or in any combination are seen to teach or suggest the Unisex External Urine Collection Apparatus of the present invention.

### SUMMARY OF THE INVENTION

In accordance with the present invention there is provided an external urine collection apparatus for use by either male or female wearers. The novel apparatus has an elongated cone-like collection apparatus having an elongated top opening forming a convex curve when viewed from the side. A conformable seal, typically fluid-filled seal, is disposed around the top opening. Strap attachment loops are disposed circumferentially around the outside surface of the urine collection apparatus. Support straps, typically four, each having a proximal end selectively attachable to and detachable from the strap attachment loops. Each attachment strap has a distal end that adjustably attaches to a waist-encircling strap or belt. These attachment straps hold the urine collection apparatus in sealed contact with the wearer's genital region. The geometry of the collection apparatus is configured to accommodate male or female genitalia. A threaded connection at the lower terminus of the elongated cone-like collection apparatus accepts a detachable drainage tube which may be connected to well-known collection bags or the like to accumulate the urine. One or more reflux valves may be located adjacent the lower terminus or in the detachable drainage hose.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a side perspective, schematic view of the unisex external urine collection apparatus with attachment straps in accordance with the present invention;

FIG. 2 is a bottom perspective, schematic view of the urine collection apparatus of FIG. 1;

FIG. 3 is a perspective, schematic view of an attachment strap for use with the urine collection apparatus of FIG. 1; and

FIG. 4 is a front perspective, schematic view of the urine collection device of FIG. 1 in its intended operating environment in position on a human wearer.

### DETAILED DESCRIPTION OF THE PREFERRED EMBODIMENT

The present invention provides a unisex external urine collection adapted for “long term” attachment to either a male or female person.

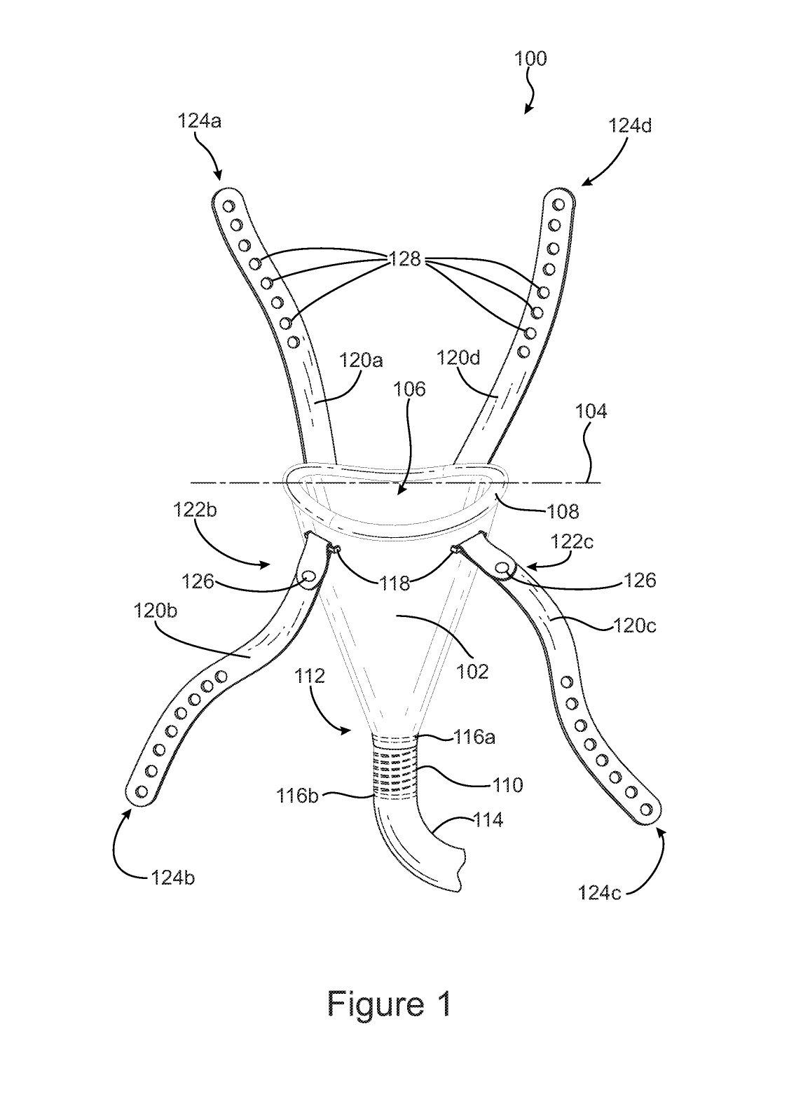

Referring first to FIG. 1, there is shown side prospective, schematic view of the unisex external urine collection apparatus with a support strap in accordance with the present invention, generally at reference number 100.

Urine collection apparatus 100 consists of an elongated cone-like collection portion 102 having an elongated opening 106 and a major axis 104 that is typically aligned front-to-back when urine collection device 100 is in place on a wearer.

Elongated opening 106 has a depressed center region and raised end regions, either specifically identified to form a concave curve thereby providing a front-to-back contour that matches the shape of a typical wearer's genital region.

An elongated, compressible seal 108 is disposed around elongated opening 106. Compressible seal 108 is typically filled with a fluid, not specifically identified. The fluid may be liquid or gaseous, neither shown. Such seals are believed to be well known to those of skill in the art and consequently, are not further discussed or described herein. Seal 108 has sufficient thickness (i.e., height) to compress to conform to minor variations in the genital anatomy of a wearer thereby sealing urine collection apparatus 100 to the wearer and ensuring minimal urine leakage.

A threaded portion 110 is formed at a narrow bottom region 112 of elongated cone-like collection portion 102. Threaded portion 110 typically has external threads, not specifically identified, adapted to receive a mating female threaded coupling of a detachable drainage hose 114. It will be recognized by those of skill in the art that alternately the threads of threaded portion 110 may be internal and the threads of the mating female coupling of detachable collection hose 114 may be external to meet a particular operating requirement. Consequently, the invention is not considered limited to a particular arrangement of male and female threads. Rather, the invention is intended to include either arrangement.

A reflux valve may be disposed either in lower portion 112 of urine collection apparatus 102 at reference number 116a or, alternately in an upper portion of detachable drainage hose 114 as shown at reference number 116b. Reflux valve 116a, 116b stops backflow of urine from detachable drainage hose 114 into lower portion 112 of urine collection apparatus 102. While reflux valves 116a, 116b are both shown in FIG. 1, it is anticipated that only one of reflux valve 116a, 116b will be installed. However, it may be desirable to have both reflux valves 116a, 116b installed to meet a specific operating requirement.

Four attachment straps 120a-120d, each have a proximal end 122a-122b and a distal end 124a-124d. Straps 120a-120d may have a plurality of holes 128 spaced apart centrally along their respective major axes.

Proximal end 122a-122d of each attachment strap 120a-120d are fastened to an attachment loop 118 affixed to elongated cone-like collection apparatus 120 and secured with a connector 126.

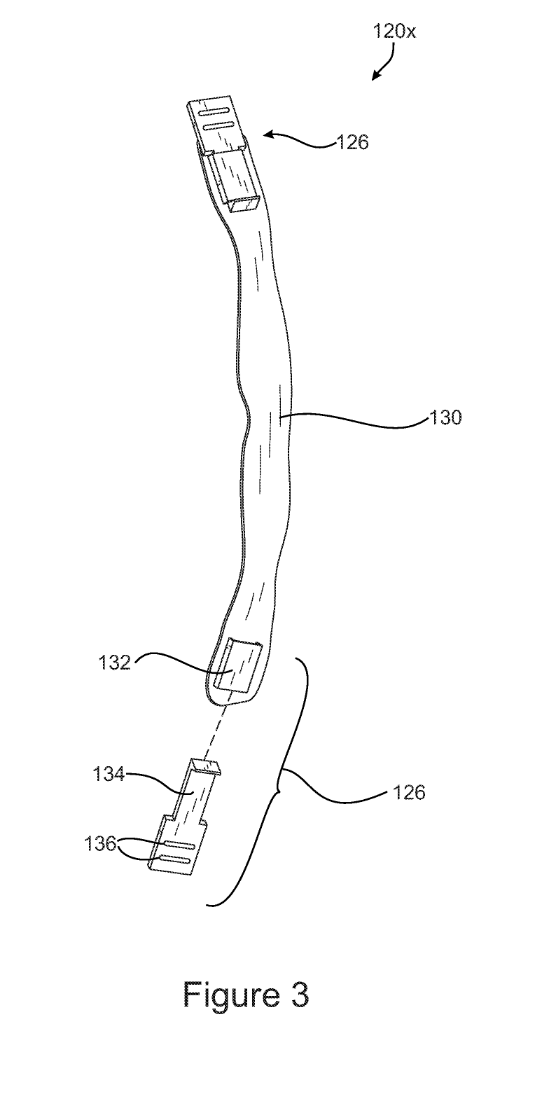

Connectors 126 may be snaps, a hook-and-loop fastening system or any other similar mechanical fasteners. A typical two-part polymeric fastener 126 suitable for the application may be seen in FIG. 3 and is discussed in more detail hereinbelow.

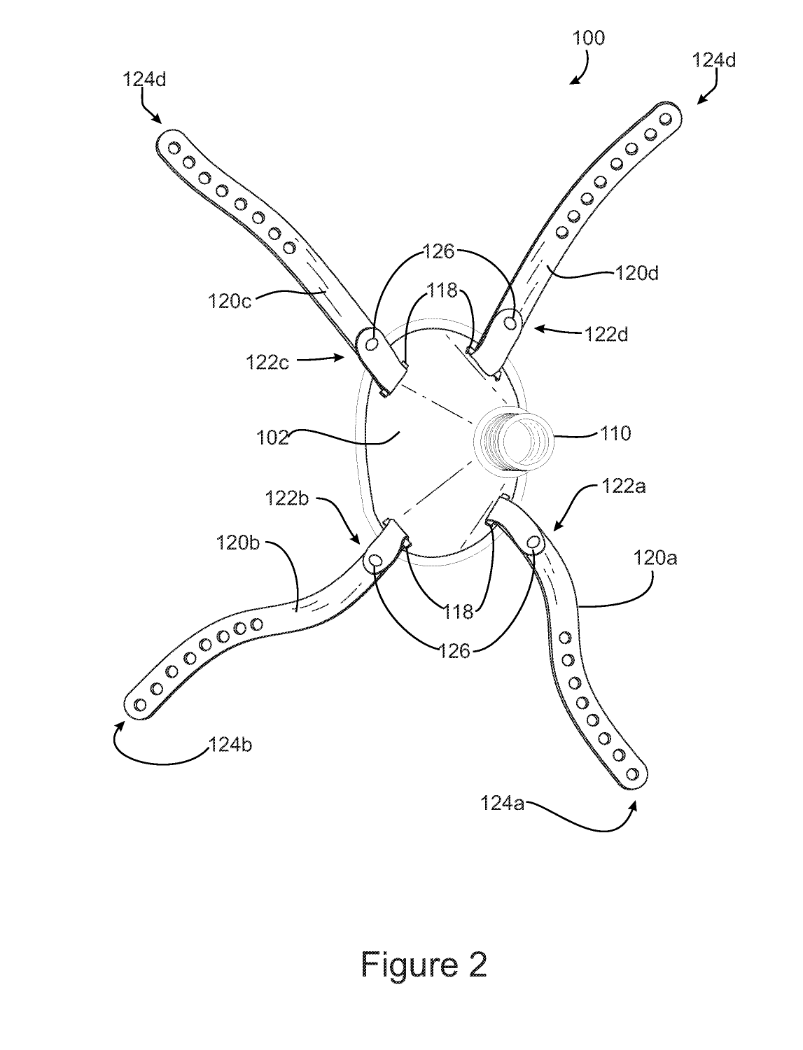

Referring now also to FIG. 2, there is shown a bottom perspective, schematic view of the elongated urine collection apparatus of FIG. 1.

Referring now also to FIG. 3, there is shown a perspective, exploded, schematic view of an attachment strap 120a-120d, generally at reference number 120x. The designation 120x is intended to represent any one of attachment straps 120a-120d.

A pliable band 130 has a two-part connector 126 formed by female component 132 and male component 134. Grooves 136 in male component 134 allow pliable band 130 bearing female component 132 to pass through. Male component 132 may then be attached to female component 132 after encircling an object such as attachment loops 118.

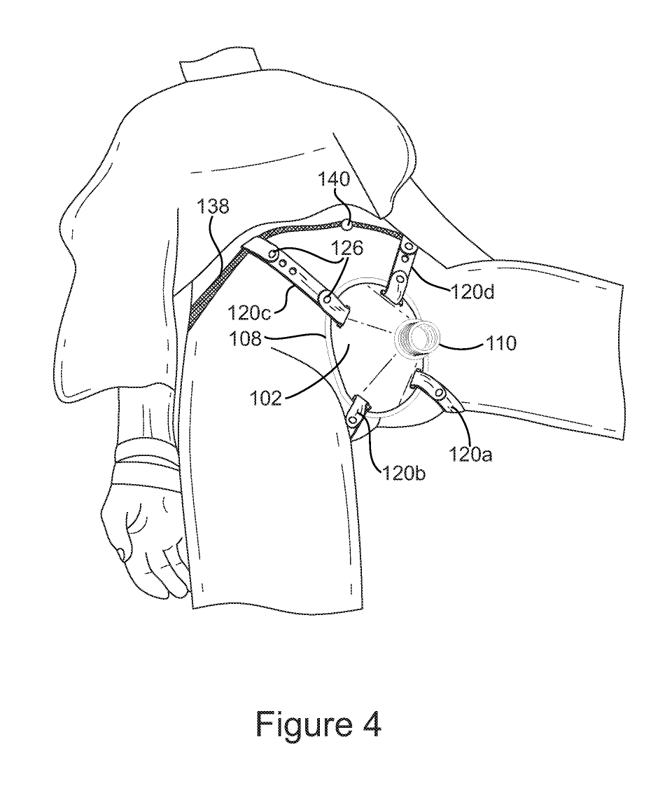

Referring now also to FIG. 4, there is shown a perspective, schematic view of the urine collection device of FIG. 1 in its intended operating position on a human patient.

Strap or belt 138 has an adjustable fastening system shown schematically at reference number 140 which is used to secure strap or belt 138 snugly around a wearer's waist. Suitable fastening systems 140 for adjustable securing strap or belt 138 are believed to be known to persons of skill in the fastening arts. Consequently, fastening systems are not further discussed herein.

Strap 120c extends upwardly across the right side of the wearer's groin. The strap region proximate distal end 124c is wrapped around strap or belt 138 that encircles the wearer's waist. Strap 120d passes upwardly across the left side of a wearer's groin and an associated distal region including distal end 124d is wrapped around strap or belt 138

Strap 120b passes through a right side of the wearer's crotch and engages strap or belt 138at the wearer's back. Similarly, strap 120a pass through a left side of the wearer's crotch and also engages strap or belt 138 at the wearer's back.

The urine collection system 100 is adapted for use by either male or female wearers. This is accomplished primarily by the geometry of elongated cone-like collection apparatus 102. It will be recognized that the urine collection apparatus may be adapted for use by only a female wearer by suitable adjustment of the geometry of the elongated cone-like collection apparatus.

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. An external urine collection device for male or female use, comprising: an elongated cone-like collection apparatus having a wide upper opening with a compliant seal circumferentially disposed thereat, said compliant seal being adapted and configured to sealably surround and engage the genital region of a wearer, said elongated cone-like collection apparatus having a narrow lower end having an open discharge region terminating in a threaded outlet tube, said urine collection apparatus having a major axis oriented along a front-to-back direction relative to said wearer.

2. The external urine collection device for male or female use as recited in claim 1, wherein said wide upper opening when viewed from the side thereof forms a concave curve.

3. The external urine collection device for male or female use as recited in claim 2, further comprising a plurality of strap connection loops disposed circumferentially around an exterior surface of said elongated cone-like collection apparatus and support straps each having a proximal end selectively attached to and detached from said plurality of strap connection loops and an opposing distal end.

4. The external urine collection device for male or female use as recited in claim 3, further comprising a belt-like structure having a proximal end and a distal end, said belt-like structure adapted to encircle a waist region of a wearer of said urine collection device and receive and adjustably retain distal regions of said narrow elongated straps thereby supporting said external urine collection apparatus in a desired position on said wearer thereof.

5. The external urine collection device for male or female use as recited in claim 4, further comprising a fastener disposed between said distal regions of each of said narrow elongated straps and said belt-like structure.

6. The external urine collection device for male or female use as recited in claim 1, further comprising a detachable drainage hose operably connected to said threaded output tube.

7. The external urine collection device for male or female use as recited in claim 6, further comprising a reflux valve disposed in at least one of the locations selected from the group: said open drainage region of said elongated cone-like collection apparatus, and said detachable drainage hose.

8. The external urine collection device for male or female use as recited in claim 1, wherein said compliant seal comprises a fluid-filled compliant seal.
